(19) United States
(12) Patent Application Publication
(10) Pub. No.: US 2014/0240359 A1
(43) Pub. Date: Aug. 28, 2014

(54) PORTABLE ANALYTE METER WITH ENHANCED MEASUREMENT DISPLAY

(71) Applicant: LIFESCAN SCOTLAND LIMITED, Inverness (GB)

(72) Inventors: David Elder, Inverness (GB); Allan Faulkner, Inverness (GB)

(73) Assignee: LifeScan Scotland Limited, Inverness (GB)

(21) Appl. No.: 13/779,135

(22) Filed: Feb. 27, 2013

(51) Int. Cl. G09G 5/38; G01N 33/66

(52) U.S. Cl. CPC G09G 5/38; G01N 33/66
USPC 345/659; 345/619; 29/825

(57) ABSTRACT

A medical testing device performs a test measurement on a sample from a user of the device and displays alphanumeric characters representative of the test measurement. The device includes a display screen integrated within the housing for displaying the alphanumeric characters when the device is held by the user in a first orientation. The alphanumeric characters are not discernable if the device is held by the user in a second orientation.

### TECHNICAL FIELD

This application generally relates to the field of blood analyte measurement systems and more specifically to a portable blood analyte meter that is configured to constrain an improper viewing orientation of the meter's display screen.

### BACKGROUND

Hand held blood analyte measurement systems are used for testing an individual's blood in a variety of surroundings at any time of day. These systems typically comprise a portable analyte meter that is configured to receive a biosensor, usually in the form of a test strip. Because these systems are portable, and testing can be completed in a short amount of time, patients are able to use such devices in the normal course of their daily lives without significant interruption to their personal routines. Therefore, a person with diabetes, for example, may measure their blood glucose levels several times a day as a part of a self management process to ensure proper control of their blood glucose within a target range.

A failure to maintain target glycemic control can result in serious diabetes-related complications including cardiovascular disease, kidney disease, nerve damage and blindness. The easier and more comfortable it is for an individual to perform blood glucose testing, the more likely that the person will be able to maintain target blood glucose levels. It is important that the use and reading of blood analyte tests be performed accurately. The display screen of a blood analyte meter should display measurement results clearly and without undue effort on the part of the user to view the display.

It is possible for a user of a portable blood analyte meter to view its display in an incorrect orientation, i.e., upside down. A built-in mechanism that encourages the user to view the meter in a correct orientation, or whose display is difficult to view or shuts off when presented in an incorrect orientation, or which automatically adjusts its display to a correct orientation may avoid an incorrect reading of the display. There currently exist a number of available portable electronic devices that can measure glucose levels in an individual based on a small sample of blood. One such analyte meter is the OneTouch® Verio™ glucose measurement system, a product which is manufactured by LifeScan, Inc.

### SUMMARY OF THE DISCLOSURE

A medical testing device may be configured several ways to facilitate correct viewing of data provided on a display screen of the device. Embodiments may include a particular shape of a housing of the meter such as top and bottom surfaces that are non-parallel, which implies, for the user, a correct orientation in viewing a display that is positioned on a top surface of the device. The display itself may also be disposed within the device housing at an angle to encourage viewing the display in a correct orientation. A polarized display screen can render higher contrast characters when the display is viewed at the correct orientation. When the screen is viewed while holding the meter at an incorrect orientation, the polarization is such that there will be insufficient contrast to read the display. In addition, provision of a position or attitude sensor, such as an accelerometer, within or on the meter can detect the orientation at which the meter is being held which may result in a corrective action such as a shutdown of the display, an error message, or wherein the display may be programmed to automatically rotate the displayed characters (typically an 180 degree rotation) to the proper orientation. A test result may be prevented from being displayed until the meter is being viewed in the correct orientation to prevent misreading of the measurement results.

In one embodiment, an apparatus with a housing encloses an electric circuit that is configured to perform a function. The apparatus has a display screen integrated within the housing for displaying a result of the function, and the result is discernable on the screen only in one orientation of the screen.

In another embodiment, a method of making an apparatus by providing a housing for retaining at least one electronic circuit is disclosed. The electronic circuit is configured for performing an electronic function. A display screen is disposed within the housing at a predetermined angle relative to a primary surface of the housing, so that the display screen is discernable based on an orientation of the housing.

In another embodiment, a method of operating an electronic device is disclosed. The device has a display screen for displaying results of measurements performed by the device. The device receives a physical sample provided to the device from a user and the device measures a property of the sample. The measured property is displayed on the display screen such that it is viewable only from one angle.

These and other embodiments, features and advantages will become apparent to those skilled in the art when taken with reference to the following more detailed modes of carrying out the invention in conjunction with the accompanying drawings that are first briefly described.

### BRIEF DESCRIPTION OF THE DRAWINGS

The accompanying drawings, which are incorporated herein and constitute part of this specification, illustrate presently preferred embodiments of the invention, and, together with the general description given above and the detailed description given below, serve to explain features of the invention (wherein like numerals represent like elements).

FIG. 1A is a perspective view of a medical testing device;

FIG. 1B illustrates a side cross-section view of the medical testing device of FIG. 1A;

FIG. 2 illustrates a side cross-section view of an alternative shape of a housing for the medical testing device of FIG. 1A;

FIG. 3A illustrates one viewing angle when using the medical testing device of FIG. 1A;

FIG. 3B illustrates another viewing angle when using the medical testing device of FIG. 1A;

FIG. 4A illustrates a polarized layer providing a viewing angle for discerning characters displayed on a display screen when viewing the medical testing device of FIG. 1A;

FIG. 4B illustrates the effect of a polarized layer to block another viewing angle when using the medical testing device of FIG. 1A;

FIG. 5A illustrates an accelerometer operative to provide a viewing angle for discerning characters displayed on a display screen when viewing the medical testing device of FIG. 1A in a first orientation;

FIG. 5B illustrates an accelerometer operative to control a display of information displayed on a display screen when viewing the medical testing device of FIG. 1A in a second orientation;

FIG. 6 illustrates a control system for operating the medical testing device of FIG. 1A; and

FIG. 7 illustrates a flow chart of a method of operating the medical testing device of FIG. 5A.

### MODES OF CARRYING OUT THE INVENTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “patient” or “user” refer to any human or animal subject and are not intended to limit the systems or methods to human use, although use of the subject invention in a human patient represents a preferred embodiment.

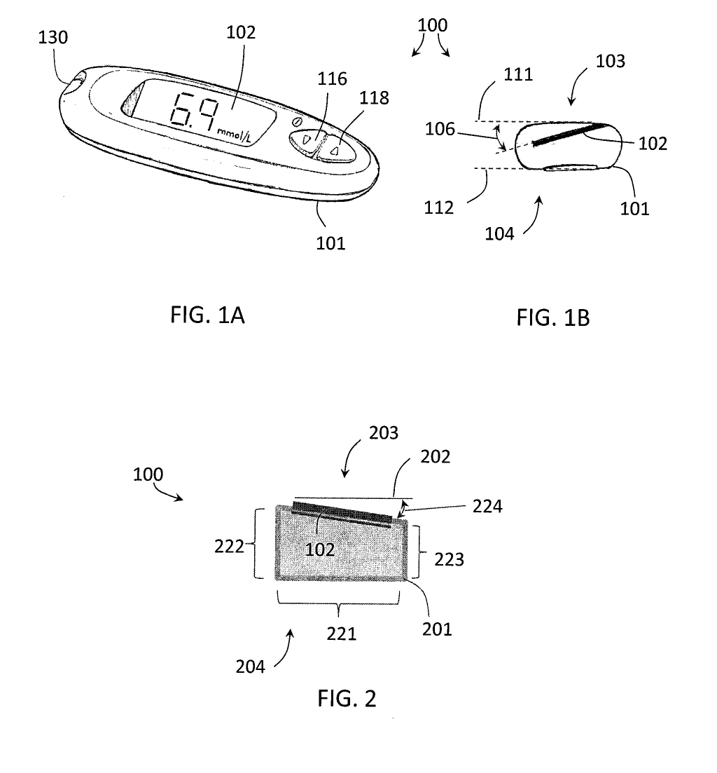

FIGS. 1A and 1B illustrate views of the medical testing device 100 for testing a blood sample provided by a user. The medical testing device 100 is defined by a housing 101 that retains electronic circuitry for managing operation of the device, as described below. The medical testing device further includes a port 130 sized for receiving a biosensor. According to this embodiment, the medical testing device 100 is a blood glucose meter and the biosensor is provided in the form of a glucose test strip. The medical testing device 100 includes a plurality of user interface buttons 116, 118, and an electronic display 102, such as an LCD display, for displaying alphanumeric characters representing a test measurement performed by the medical testing device 100 on a blood sample provided by a user of the medical testing device 100. The plurality of user interface buttons 116, 118 can be configured to prompt an output of data and to navigate options presented on the display 102, and to execute commands. Output data can include alphanumeric values representative of a blood analyte concentration presented on the display 102.

The housing 101 of the medical testing device 100 includes a top surface 103 in a plane 111 substantially parallel to a plane 112 of the bottom surface 104 of the housing 101. The display screen 102 in this exemplary embodiment is mounted at an angle 106 of approximately 15 to 25 degrees relative to either of the planes defined by the top surface 111 or of the bottom surface 112 of the housing 101. The display screen 102 is integrated within said housing 101 at the predetermined mounting angle to facilitate viewing the display screen 102 in a proper orientation for reading the alphanumeric characters presented on display screen 102, as described below.

FIG. 2 illustrates a side cross-section view of an alternative configuration for the housing 201 of the medical testing device 100 as described above. The display screen 102 in this exemplary embodiment is mounted in a plane which is coplanar, or substantially parallel, to the top surface 203 of the housing 201. In this exemplary embodiment, the top surface 203 is not parallel to the bottom surface 204. The cross section of the housing 201 of the exemplary medical testing device 100 comprises a trapezoidal shape that may have a bottom dimension 221 of about 30 mm, two side dimensions 222, 223, of unequal lengths 20 mm and 15 mm, respectively, each extending substantially orthogonally from the bottom surface 204, and a top surface 203 connected to the two sides and tilted at an angle 224 of approximately 15 to 25 degrees relative either to the bottom surface 204 or to a horizontal plane 202 substantially parallel to the bottom surface 204 of the housing 201. A length of the medical testing device (into the page) is approximately 100 mm. Each of the foregoing dimensions is exemplary and can be scaled effectively. Similar to the orientation strategy of the display screen 102 described above with reference to FIGS. 1A-1B, the display screen 102 is integrated within the top surface 203 of the housing 201 and at a predetermined mounting angle to facilitate viewing the display screen 102 in a proper orientation for reading the alphanumeric characters presented thereon, as will now be described.

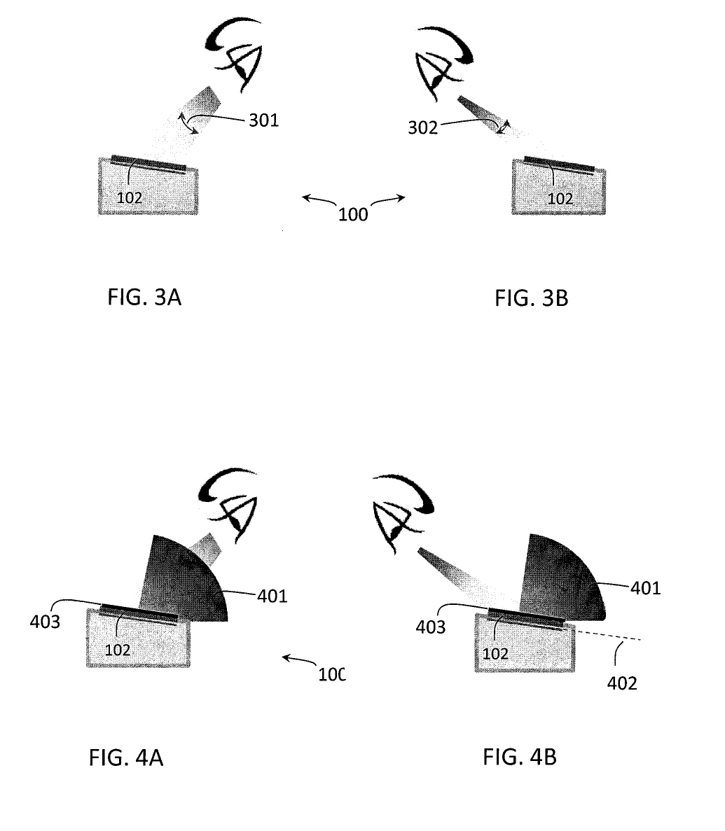

FIGS. 3A-3B illustrate that the medical testing device 100 having an angled display screen 102, in either of the embodiments of FIG. 1A or FIG. 2, can be viewed in at least two orientations. More specifically, a first defined orientation wherein the medical testing device 100 is oriented so that display screen 102 is tilted toward the user, FIG. 3A, shows that the screen 102 provides a wider field of view 301, as compared with a second orientation, FIG. 3B, wherein the viewing angle 302 is more acute, thereby narrower than the viewing angle 301, and the alphanumeric characters on display screen 102 are much less discernable. Thus, the alphanumeric characters are more easily discernible in one of said orientations, namely, the orientation illustrated in FIG. 3A because of the user's wider viewing angle 301. Thus, the shape of the housing 201 of the medical testing device 100, or an angle of the display screen mounted in the housing 101 (FIG. 1A) serves to influence a user to orient the device in the proper first orientation during use such as, for example, if the device was positioned on a table top. It should be noted that the following embodiments, though described with reference to the trapezoidal cross-section embodiment illustrated in FIG. 2, apply equally to the medical testing device embodiment illustrated in FIG. 1A.

FIGS. 4A-4B illustrate another embodiment of a medical testing device 100 having a similar overall structure as described above. Electronic display screen 102, such as an LCD screen, for displaying alphanumeric characters comprises a viewing angle control polarizer, that is oriented over, on, or built into, the display screen 102 such that the alphanumeric characters are discernible in only one of said first and second orientations, namely, the first orientation as illustrated in FIG. 4A. This is because the polarized layer over the display screen 102 is disposed such that it provides a viewing angle 401 that blocks a user from discerning alphanumeric characters on the display screen 102 when viewed in a direction outside of the viewing angle 401, such as from the angle as illustrated in FIG. 4B. The polarized layer 403 is selected to allow a viewing angle ranging from about five degrees to about eighty-five degrees relative to the plane of the display screen 102. Thus, the polarized layer 403 serves to insure that the user is reading the display screen 102 in the proper orientation. Polarized display manufacturers that may provide suitable polarized display screens as described herein include: Yeebo (International Holdings) Ltd. 7/F, On Dak Industrial Building, 2-6 Wah Sing Street, Kwai Chung, New Territories, Hong Kong; and Shenzen AV Display Co. Ltd. 39, Jinyuan Road, He'ao Jinyuan Industrial Zone, Henggang, Shenzhen, China. Manufacturers of polarized film include Nikko Denko Corporation. Umeda, Kita-ku, of Osaka, Japan, among others.

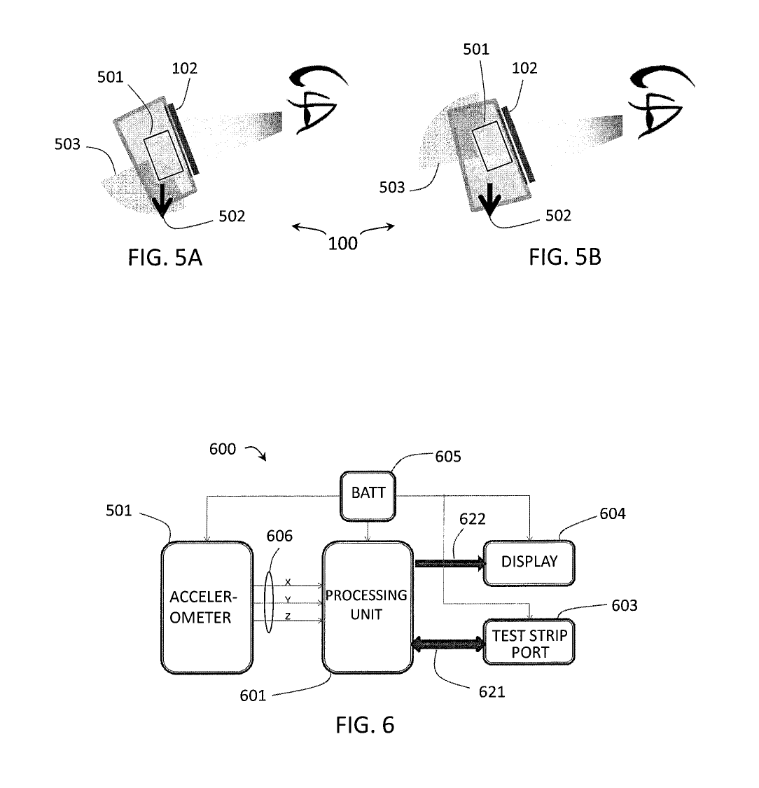

FIGS. 5A-5B illustrate another embodiment of a medical testing device 100 having a similar overall structure as described above. The medical testing device 100 comprises an electronic display screen 102, such as an LCD screen, for displaying alphanumeric characters, and at least one position or attitude sensor, such as an accelerometer 501 for detecting an inclination, or orientation, of the medical testing device with reference to the direction of the earth's gravitational pull 502 (acceleration due to gravity). According to the exemplary embodiment, a two or three axis accelerometer 501 is used, although multiple single axis accelerometers and/or combinations thereof can be used wherein the accelerometers can be analog or digitally based. In terms of operation, the accelerometer 501 transmits data corresponding to the orientation angle of the medical testing device 100 with reference to direction 502 to a processing unit, such as a microcontroller, which compares the received orientation angle with an acceptable range of orientation angles stored in the microcontroller, and illustrated in FIGS. 5A and 5B as the range of acceptable orientation angles 503, to determine if the current orientation of the device 100 is properly within the orientation angle range 503. If the accelerometer 501 transmits data indicating that the current orientation angle of the device 100 places the reference direction 502 within the orientation angle range 503, as illustrated in FIG. 5A, the medical testing device 100 operates in a normal fashion during use by displaying alphanumeric characters on the display screen 102 representative of a measured blood analyte level. If the accelerometer 501 transmits data indicating that the current orientation angle of the device does not place the reference direction 502 within the orientation angle range 503, as illustrated in FIG. 5B, the microcontroller may be programmed to respond in a variety of ways. One embodiment may include a programmed algorithm stored in the microcontroller that causes the display screen 102 to be turned off, thereby preventing a display of the alphanumeric characters thereon. Another embodiment may include a programmed algorithm stored in the microcontroller that causes an error message to be displayed on the display screen 102. A further embodiment may include a programmed algorithm stored in the microcontroller that causes the alphanumeric characters displayed on the display screen 102 to be inverted by rotating the alphanumeric characters 180 degrees on the display screen 102. Accelerometers that provide sufficient inclination detection as described above are identified as follows: analog accelerometer ADXL330 from Analog Devices; analog accelerometer MMA7341LC from Freescale; and digital accelerometer LIS35DE from ST Microelectronics.

Referring to FIG. 6, the control system 600 of the medical testing device 100 may be disposed on, for example, a printed circuit board situated within the housing 101. FIG. 6 illustrates, in simplified schematic form, several of the electronic components disposed within the housing 101 for purposes of controlling operations of the medical testing device as described herein. The control system 600 includes a processing unit 601 in the form of a microprocessor, a microcontroller, an application specific integrated circuit (“ASIC”), a mixed signal processor (“MSP”), a field programmable gate array (“FPGA”), or a combination thereof, comprising memory sufficient for storing programmed control operations as described herein, and is electrically connected to various electronic modules and components included on, or connected to, the printed circuit board. The processing unit 601 is electrically connected to, for example, a test strip port circuit 603 via a communication line 621. The strip port circuit 603 is electrically connected to the test strip port 130 during blood glucose testing. To measure analyte concentration, the strip port circuit 603 detects a current across biased electrodes of an analyte test strip having a blood sample disposed thereon, using a potentiostat, and converts an electric current measurement into digital form for presentation on the display 102. The processing unit 601 can be configured to receive input from the strip port circuit 603 and may also perform a portion of the potentiostat function and the current measurement function. The analyte test strip can be in the form of an electrochemical glucose test strip.

A display module 604, which may include a display processor and display buffer, is electrically connected to the processing unit 601 over the communication line 622 for receiving and displaying analyte measurement data in, for example, alphanumeric form, or for displaying messages transmitted thereto by the processing unit. The processing unit may also power off/on the display in response to program control. A battery 605 is electrically connected to all components within the housing and to the processing unit 601 to supply electric power thereto. The battery 605 may comprise standard or rechargeable batteries. The battery 605 is also electrically connected to the processing unit 601 such that processing unit 122 can monitor a power level remaining in the battery. An example operation of accelerometer 501 useful for the various embodiments described herein has been explained with reference to FIGS. 5A and 5B above. The accelerometer may comprise a three-axis analog accelerometer providing data over accelerometer output lines 606 to processing unit 601, or it may comprise a single-axis output (gravitational inclination), or a digital output.

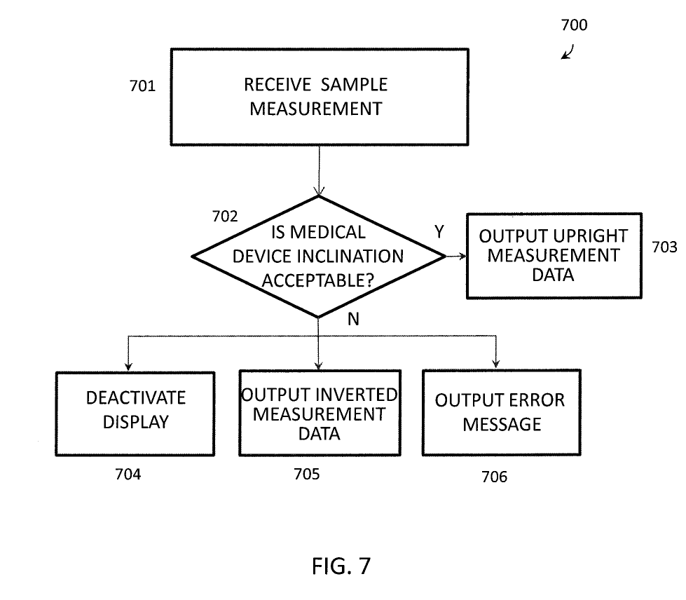

With reference to FIG. 7, there is illustrated a programmed method of operating the medical testing device 100 that utilizes several of its features just described with reference to the accelerometer embodiment as illustrated in FIGS. 5A and 5B. At step 701, the processing unit receives measurement data indicating an analyte level of a blood sample provided to the test strip port of the medical testing device. Prior to displaying the alphanumeric measurement data on the display screen representative of the analyte level, the processing unit receives accelerometer output data indicating an inclination of the medical testing device and determines whether the device's inclination is within an acceptable range, at step 702. If the inclination angle is within an acceptable range, the processing unit outputs the alphanumeric data representative of the analyte level to the display screen for viewing by the user, at step 703, in a normal upright format. If the inclination angle is determined not to be within an acceptable range at step 702, the processing unit operates in one of several alternative programmed methods. A first selectable programmed alternative, at step 704, is for the processing unit to deactivate, or turn off, the display to prevent viewing of alphanumeric data representative of the analyte level on the display screen. A second selectable programmed alternative, at step 705, is for the processing unit to output the alphanumeric characters representative of the analyte level in an inverted format, i.e. rotated 180 degrees, on the display screen. A third selectable programmed alternative, at step 706, is for the processing unit to output an error message on the display, or a display message indicating that the medical testing device should be rotated to a correct orientation.

As will be appreciated by one skilled in the art, aspects of the present invention may be embodied as a system, method, or computer program product. Accordingly, aspects of the present invention may take the form of an entirely hardware embodiment, an entirely software embodiment (including firmware, resident software, micro-code, etc.), or an embodiment combining software and hardware aspects that may all generally be referred to herein as a “circuit,” “circuitry,” “component’, “module,” and/or “system.” Furthermore, aspects of the present invention may take the form of a computer program product embodied in one or more computer readable medium(s) having computer readable program code embodied thereon.

### PARTS LIST FOR FIGS. 1-7

100 medical testing device
101 housing
102 display screen
103 housing top surface
104 housing bottom surface
106 angle of display screen
111 plane of top surface of device
112 plane of bottom surface of device
116 button
118 button
130 test strip port
201 housing
202 plane parallel to bottom surface
203 alternative housing top surface
204 alternative housing bottom surface
221 base dimension
222 first side dimension
223 second side dimension
224 angle of display screen
301 width of viewing angle
302 width of viewing angle
401 polarized viewing angle
402 plane of display screen
403 polarized film
501 accelerometer
502 downward orientation
503 orientation angle range
600 medical testing device control system
601 processing unit
603 test strip port circuit
604 display circuit
605 battery
606 accelerometer output lines
621 processing unit communication line
622 processing unit communication line
700 method of operating accelerometer driven medical testing device
701 step—receive sample measurement
702 decision step—is medical device upright
703 step—output upright measurement data
704 step—deactivate display
705 step—output inverted measurement data
706 step—output error message

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well.

What is claimed is:

1. An apparatus comprising:
a housing enclosing an electric circuit configured for performing a function; and
a display screen integrated within said housing for displaying a result of the function, said result screen being discernable only in one orientation of the display screen.

2. The apparatus of claim 1, wherein the apparatus comprises a medical device for receiving a sample from a user of the medical device, the result of the function comprises alphanumeric characters representative of a test measurement of the sample, the electric circuit performs the test measurement of the sample, and wherein the display screen is viewable in a plurality of orientations.

3. The apparatus of claim 2, wherein said display screen is oriented at a preselected angle relative to a primary surface of said housing.

4. The apparatus of claim 2, wherein the first and second orientations are 180 degrees when the device is rotated in a plane defined by the primary surface of said housing.

5. The apparatus of claim 2, further comprising at least one polarized layer disposed over the display screen.

6. The apparatus of claim 5, wherein the polarized layer is configured to provide a limited viewing angle of the display screen within which the alphanumeric characters are discernible and outside of which the alphanumeric characters are not discernible.

7. The apparatus of claim 6, wherein the discernable viewing angle of the display screen is between about five degrees and about eighty-five degrees relative to a plane of the display screen.

8. The apparatus of claim 5, wherein the polarized layer is a polarized film.

9. The apparatus of claim 2, further comprising at least one accelerometer for detecting the orientation of the medical device.

10. The apparatus of claim 9, further comprising a circuit connected to the at least one accelerometer for automatically turning off the display screen when the device is in the non-discernable orientation.

11. The apparatus of claim 9, further comprising a circuit connected to the at least one accelerometer for displaying an error message on the display screen when the device is held by the user in the non-discernable orientation.

12. The apparatus of claim 9, further comprising a circuit connected to the accelerometer for displaying the numerical characters inverted on the display screen when the device is held by the user in the non-discernable orientation.

13. The apparatus of claim 2, wherein the display screen comprises a liquid crystal display screen.

14. The apparatus of claim 2, wherein the medical device is a blood glucose meter.

15. A method of manufacturing an apparatus, the method comprising:
providing a housing for retaining at least one electronic circuit configured for performing an electronic function; and
disposing a display screen within said housing at a predetermined angle relative to a primary surface of said housing, such that said display screen is discernable based on an orientation of said housing.

16. The method of claim 15, wherein the apparatus comprises a medical device, the method further comprising:
the electronic circuit testing a sample from a user of the medical device; and
displaying on the display screen alphanumeric characters representative of a test measurement of the sample, wherein the predetermined angle enables said alphanumeric characters to be readable in only in one orientation of the housing.

17. The method of claim 16, further comprising:
providing at least one accelerometer relative to said housing for detecting said orientation of said housing.

18. The method of claim 17, further comprising:
providing a controller within said housing, the controller electrically connected to said display screen for controlling the alphanumeric characters displayed on the display screen; and
configuring said controller to turn off the display screen in response to receiving an output from the accelerometer indicating that the orientation of said housing causes the display screen to be non-discernable.

19. The method of claim 17, further comprising:
providing a controller within said housing, the controller electrically connected to said display screen for controlling the alphanumeric characters displayed on the display screen; and
configuring said controller to display an error message on the display screen in response to receiving an output from the accelerometer indicating that the orientation of said housing causes the display screen to be non-discernable.

20. The method of claim 17, further comprising:
providing a controller within said housing, the controller electrically connected to said display screen for controlling the alphanumeric characters displayed on the display screen; and
configuring said controller to invert the alphanumeric characters to be displayed on the display screen in response to receiving an output from the accelerometer indicating that the orientation of said housing causes the display screen to be non-readable.

21. The method of claim 16, wherein a first orientation of the housing allows the user to view the display screen at a wider angle than a second orientation.

22. The method of claim 16, further comprising:
disposing a polarized layer over the display screen, the polarized layer blocking the alphanumeric characters from view in a first orientation of the housing and not blocking the alphanumeric characters from view in a second orientation of the housing.

23. The method of claim 22, further comprising configuring the polarized layer such that said second orientation includes a viewing angle of between about five degrees and eighty-five degrees relative to a plane of the display screen.

24. The method of claim 16, wherein the step of the electronic circuit testing the sample from the user comprises determining a glucose level of a blood sample.

25. A method of operating an electronic device having a display screen for displaying results of measurements performed by the device, the method comprising:
receiving a physical sample provided to the device from a user;
measuring a property of the sample; and
displaying the measured property on the display screen such that it is viewable only from a first angle.

26. The method of claim 25, wherein the device is a medical device, the method further comprising:
measuring an analyte level in the sample; and
preventing the user from viewing the measured analyte level on the display screen when the user views the display screen from a second angle.

27. The method of claim 26, further comprising not preventing the user from viewing the measured analyte level on the display screen when the user views the display screen from the first angle.

28. The method of claim 27, further comprising:
displaying the measured analyte level on the display screen using alphanumeric characters; and
blocking from view the alphanumeric characters when the display screen is viewed from the second angle.

29. The method of claim 28, wherein the step of blocking includes disposing a polarized filter over the display screen.

30. The method of claim 29, further comprising configuring the polarized filter such that the first angle ranges from between about five degrees and about eighty-five degrees relative to a plane of the display screen.

31. The method of claim 29, wherein the step of disposing a polarized filter over the display screen comprises disposing a polarized film or layer directly on the display screen.

32. The method of claim 26, further comprising:
determining a spatial orientation of the medical device using at least one accelerometer;
determining that the user is viewing the display screen from the second angle based on the step of determining the spatial orientation; and
turning off the display screen in response to the step of determining that the user is viewing the display screen from the second angle.

33. The method of claim 26, further comprising:
determining a spatial orientation of the medical device using at least one accelerometer;
determining that the user is viewing the display screen from the second angle based on the step of determining the spatial orientation; and
displaying an error message on the display screen in response to the step of determining that the user is viewing the display screen from the second angle.

34. The method of claim 26, wherein the step of measuring the analyte level in the sample comprises measuring a glucose level in a blood sample.
